(19) United States
(12) Patent Application Publication
(10) Pub. No.: US 2026/0221283 A1
(43) Pub. Date: Jul. 30, 2026

(54) SYSTEM AND METHOD FOR ORGANELLOME-BASED BIOLOGICAL SAMPLE ANALYSIS

(71) Applicant: YEDA RESEARCH AND DEVELOPMENT CO. LTD., Rehovot (IL)

(72) Inventors: Eran HORENSTEIN, Tel Aviv Yaffo (IL); Lena MOLITOR, Rehovot (IL); Welmoed VAN ZUIDEN, Rehovot (IL); Nancy-Sarah YACOVZADA, Rehovot (IL); Sagy KRISPIN, Rehovot (IL); Yehuda Matan DANINO, Rehovot (IL); Noam RUDBERG, Rehovot (IL)

(21) Appl. No.: 19/459,126

(22) Filed: Jan. 26, 2026

Related U.S. Application Data
(60) Provisional application No. 63/749,910, filed on Jan. 27, 2025.

(51) Int. Cl. G16H 50/20; G06T 7/00; G16H 20/10; G16H 30/40; G16H 50/70

(52) U.S. Cl. CPC G16H 50/20; G06T 7/0014; G16H 20/10; G16H 30/40; G16H 50/70; G06T 2207/10056; G06T 2207/20081; G06T 2207/30024

(57) ABSTRACT

The present disclosure provides a method of determining a condition of a biological sample by at least one processor. The method comprises receiving one or more target microscopy images associated with the biological sample, wherein each microscopy image depicts cellular organelles pertaining to specific organelle types. The method further comprises inferring a Vision Transformer (ViT) model on the one or more target microscopy images, to generate, for each organelle type, a corresponding embedding vector, wherein the ViT model is trained using contrastive learning to distinguish between different perturbations of the organelles. The method additionally comprises applying at least one analysis model on the embedding vectors, to predict the condition of the biological sample. The predicted condition may include disease states, cellular responses, genetic mutation status, developmental stages, metabolic states, or organelle dysfunction states.

### CROSS-REFERENCE TO RELATED APPLICATIONS

This application claims benefit of priority of U.S. Patent Application No. 63/749,910, filed Jan. 27, 2025, which is hereby incorporated by reference in its entirety.

### FIELD OF THE INVENTION

The present invention relates to systems and methods for analyzing microscopy images of biological samples, and more particularly to organellome-based biological sample analysis.

### BACKGROUND

Organellome analysis involves the comprehensive study of cellular organelles, their structures, functions, and interactions within living cells. This field of research provides valuable insights into cellular biology, disease mechanisms, and potential therapeutic targets. By examining the entire complement of organelles within a cell, researchers can gain a holistic understanding of cellular processes and how they may be affected by various conditions or perturbations.

Current technologies for organellome analysis include biochemical fractionation, microscopy-based imaging, and mass spectrometry-based proteomics. Biochemical fractionation involves isolating individual organelles through centrifugation and density gradient separation techniques. Microscopy-based imaging utilizes various forms of microscopy, such as confocal and electron microscopy, to visualize organelles and their spatial relationships within cells. Mass spectrometry-based proteomics allows for the identification and quantification of proteins associated with specific organelles.

A common challenge across these methods is the difficulty in integrating data from multiple organelles simultaneously. Many existing approaches focus on studying individual organelles in isolation, which may not fully capture the interconnected nature of cellular processes and organelle interactions. This limitation can hinder our ability to understand complex cellular responses to perturbations or disease states.

Given this limitation, there is a clear need for improved methods of organellome analysis that can provide a more comprehensive, integrated view of cellular organelles and their functions. Advancements in this field could potentially enhance our understanding of cellular biology, disease mechanisms, and drug responses, ultimately contributing to the development of more effective diagnostic tools and therapeutic strategies.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

Embodiments of the present invention relate to a method and system for determining a condition of a biological sample using advanced image analysis techniques. Embodiments of the invention may utilize a Vision Transformer (ViT) model trained with a contrastive learning framework, to analyze microscopy images of cellular organelles and predict various biological conditions.

Embodiments of the present invention may employ the ViT model to generate embedding vectors for different organelle types. The application of contrastive learning may serve to produce embedding vectors that distinguish between different perturbations of organelles. These embedding vectors may be further analyzed to predict a wide range of biological sample conditions, including for example disease states, cellular stress responses, drug responses, genetic mutation status, and the like.

Embodiments of the invention may incorporate a pre-training step on non-perturbed biological samples, to establish baseline embeddings, followed by fine-tuning on perturbed samples to generate perturbation-specific embeddings. The fine-tuning process employs the contrastive learning framework to minimize variation between similar perturbations while accentuating differences between distinct perturbations.

As elaborated herein, embodiments of the invention may combine embeddings from different organelle types, to create an integrated organellome view, and perform cluster analysis of these integrated views to identify perturbation patterns.

Embodiments of the invention may also rank organelles to quantify changes in response to perturbations. Such ranking may be utilized to identify potential drug targets based on the analysis of organelle relevance to specific diseases or conditions.

This innovative approach to biological sample analysis offers improved accuracy and depth of insight compared to conventional methods, making it particularly suitable for advanced diagnostic applications, drug discovery, and personalized medicine.

According to an aspect of the present disclosure, a method of determining a condition of a biological sample by at least one processor is provided. The method comprises receiving one or more target microscopy images associated with the biological sample, wherein each microscopy image depicts cellular organelles pertaining to specific organelle types. The method comprises inferring a Vision Transformer (ViT) model on the one or more target microscopy images, to generate, for each organelle type, a corresponding embedding vector, wherein the ViT model is trained using contrastive learning to distinguish between different perturbations of the organelles. The method comprises applying at least one analysis model on said embedding vectors, to predict the condition of the biological sample.

According to other aspects of the present disclosure, the method may include one or more of the following features. The predicted condition of the biological sample may be selected from a list consisting of: a diagnosis of a disease state, a prognosis of a disease state, a cellular stress response, a drug response, a genetic mutation status, a developmental stage, a metabolic state, an activation state of a cellular pathway, a differentiation state of cells, an aging-related state, a cell cycle phase, an inflammatory state, a protein aggregation state, an organelle dysfunction state. The method may further comprise pre-training the ViT model on a first dataset of microscopy images, depicting non-perturbed biological samples, to generate baseline embedding vectors, encoding states of organelles of respective types in said non-perturbed biological samples.

The organelle states may be selected from a list consisting of image-observable proxies of localization patterns of respective organelles within the microscopy images, morphological patterns of the respective organelles, properties of functionality of the respective organelles, and any combination thereof. The organelle states may be selected from a list consisting of: localization patterns of the respective organelles within the microscopy images, morphological patterns of the respective organelles, size variations of the respective organelles, shape alterations of the respective organelles, distribution patterns of the respective organelles within cells, clustering or dispersion of the respective organelles, interactions between different types of organelles, membrane integrity of the respective organelles, protein composition of the respective organelles, lipid composition of the respective organelles, enzymatic activity within the respective organelles, oxidative stress markers in the respective organelles, organelle-specific protein aggregation, organelle-specific gene expression profiles, post-translational modifications of organelle-associated proteins, and any combination thereof.

The method may further comprise obtaining a second dataset of microscopy images, each (a) depicting organelles of a specific type, extracted from a perturbed biological sample, and (b) annotated according to said perturbation. The method may comprise using the image annotations as supervisory data, to fine-tune the ViT model based on microscopy images of the second dataset, thereby generating an operational version of the ViT model, wherein said operational version is configured to generate perturbation embeddings that associate states of organelles of specific types with respective biological sample perturbations. The perturbations may comprise null perturbations, disease-related perturbations and experimental manipulations of the biological sample. The perturbations may comprise null perturbations, disease-related perturbations, genetic modifications, drug treatments, environmental stress conditions, metabolic alterations, cellular differentiation processes, and aging-related changes.

Fine-tuning the ViT model may comprise selecting one or more tuples of microscopy images of the second dataset, wherein each tuple's microscopy images depict organelles of a specific type, originating from biological samples that pertain to two or more different perturbations. Fine-tuning may comprise applying a contrastive learning framework on the one or more tuples, to calculate a contrastive loss function that is adapted to (i) minimize variation between perturbation embeddings pertaining to similar perturbations, and (ii) accentuate variation between perturbation embeddings pertaining to different perturbations. Fine-tuning may comprise updating parameters of the ViT model by backpropagating gradients computed from the contrastive loss function. Each tuple may comprise: (i) an anchor image depicting an organelle of a specific type pertaining to a first perturbation; (ii) a positive image depicting another organelle of the same specific type, pertaining to the first perturbation; and (iii) a negative image depicting another organelle of the same specific type pertaining to a second, different perturbation.

The at least one analysis model may comprise a Machine-Learning (ML) based classification model, trained to receive one or more perturbation embeddings, originating from said target microscopy images as embedding vectors, and predict the condition of the biological sample based on said embedding vectors. The at least one analysis model may be configured to combine perturbation embeddings that pertain to different organelle types, but represent a common perturbation, to generate a superposition vector, and reduce a dimension of the superposition vector, to generate a compact superposition vector, representing an integrated organellome view of a specific biological sample perturbation. The at least one analysis model may be further configured to obtain a plurality of compact superposition vectors, pertaining to a respective plurality of biological samples, and group the plurality of compact superposition vectors into clusters of a clustering model, wherein each cluster describes a perturbation of respective biological samples.

The method may further comprise obtaining a target compact superposition vector, originating from the one or more target microscopy images of the target biological sample, associating the target compact superposition vector with a specific cluster of the clustering model, based on a predetermined distance metric, and determining the condition of the biological sample based on said association. The method may further comprise identifying a treatment-related perturbation that reverses a disease-related perturbation of a specific disease in a vector space of the clustering model, wherein the treatment-related perturbation is associated with administration of a substance to the biological sample, and designating the substance for drug development, for treating said specific disease.

The at least one analysis model may comprise an organelle ranking module, configured to, based on the perturbation embeddings, quantify changes in states of organelles of different types, in response to respective biological sample perturbations, and rank the organelle types according to discriminatory power between said perturbations, based on said quantification. The method may further comprise identifying a subset of organelle types with rankings above a predetermined threshold, analyzing the subset of organelle types to determine their relevance to a specific disease or condition, and designating at least one organelle type from the analyzed subset as a drug development target based on its relevance to the specific disease or condition and its discriminatory power as indicated by the ranking.

According to another aspect of the present disclosure, a system for determining a condition of a biological sample is provided. The system comprises a non-transitory memory device, wherein modules of instruction code are stored, and at least one processor associated with the memory device, and configured to execute the modules of instruction code. Upon execution of said modules of instruction code, the at least one processor is configured to receive one or more target microscopy images associated with the biological sample, wherein each microscopy image depicts cellular organelles pertaining to specific organelle types. The at least one processor is configured to infer a Vision Transformer (ViT) model on the one or more target microscopy images, to generate, for each organelle type, a corresponding perturbation embedding, wherein the ViT model is trained using contrastive learning to distinguish between different perturbations of the organelles. The at least one processor is configured to apply at least one analysis model on said perturbation embeddings, to predict the condition of the biological sample.

According to other aspects of the present disclosure, applying the at least one analysis model may comprise combining perturbation embeddings that pertain to different organelle types, but represent a common perturbation, to generate a superposition vector. Applying the at least one analysis model may comprise reducing a dimension of the superposition vector, to generate a target compact superposition vector, representing an integrated organellome view of the target biological sample perturbation. Applying the at least one analysis model may comprise obtaining a clustering model comprising a plurality of clusters of compact superposition vectors, originating from a cohort of biological samples, wherein each cluster pertains to a specific perturbation. Applying the at least one analysis model may comprise associating the target compact superposition vector with a specific cluster of the clustering model, based on a predetermined distance metric. Applying the at least one analysis model may comprise determining the condition of the biological sample based on said association.

Embodiments of the invention may include a method of identifying a drug target for treating a disease by at least one processor. Embodiments of the method may include receiving a plurality of microscopy images associated with a cohort of biological samples, where each microscopy image depicts cellular organelles pertaining to specific organelle types. The cohort may include biological samples associated with a healthy state and biological samples associated with a diseased state.

According to some embodiments, the at least one processor may apply, or infer a Vision Transformer (ViT) model on the plurality of microscopy images, to generate, for each organelle type, corresponding perturbation embeddings. The ViT model may be trained using contrastive learning to distinguish between different perturbations of the organelles.

The at least one processor may combine perturbation embeddings that pertain to different organelle types but represent a common perturbation, to generate superposition vectors, and may reduce dimension of the superposition vectors, to generate compact superposition vectors representing integrated organellome views of respective biological sample perturbations. The at least one processor may proceed to group the compact superposition vectors into clusters of a clustering model, wherein each cluster pertains to a specific perturbation (e.g., a healthy state and a diseased state).

The at least one processor may calculate a disease-related perturbation vector in a vector space of the clustering model, originating from a cluster associated with the healthy state and terminating at a cluster associated with the diseased state. The at least one processor may subsequently identify a treatment-related perturbation whose superposition vector amplitude substantially matches an amplitude of the disease-related perturbation vector, but has an opposite direction. The treatment-related perturbation may, for example, be associated with administration of a substance to a biological sample. The at least one processor may subsequently designate (e.g., produce an appropriate notification that designates) the treatment (e.g., the administered substance) as a drug target for treating the disease.

The foregoing general description of the illustrative embodiments and the following detailed description thereof are merely exemplary aspects of the teachings of this disclosure and are not restrictive.

### BRIEF DESCRIPTION OF FIGURES

The patent or application file contains at least one drawing executed in color. Copies of this patent or patent application publication with color drawing(s) will be provided by the Office upon request and payment of the necessary fee.

Non-limiting and non-exhaustive examples are described with reference to the following figures.

FIG. 1 is a block diagram depicting a computing device that may be used within a system for determining a condition of a biological sample based on microscopy images, according to some embodiments of the invention;

FIG. 2 is a block diagram illustrating a system for analyzing microscopy images to determine biological conditions and potential drug targets, according to some embodiments of the invention;

FIG. 3 is a block diagram showing operation of the system during a training stage, according to some embodiments of the invention;

FIG. 4 is a block diagram depicting operation of a sample and match module that may be included in the system for processing cell images and annotation data, according to some embodiments of the invention;

FIG. 5 is a block diagram illustrating a contrastive learning dataflow, which may be employed by the system during a training stage, according to some embodiments of the invention;

FIG. 6 is a bubble diagram, showing experimental results, by which organelle scores were calculated for different organelle types, in relation to different types of perturbations, according to some embodiments of the invention;

FIG. 7A is a schematic diagram showing workflow of a biological condition analysis module which may be included in the system according to some embodiments of the invention;

FIG. 7B is a scatter graph, showing experimental results for predicting a condition of a biological sample according to some embodiments of the invention; and

FIG. 8 is a flowchart showing a method for determining a condition of a biological sample using microscopy images, according to some embodiments of the invention.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

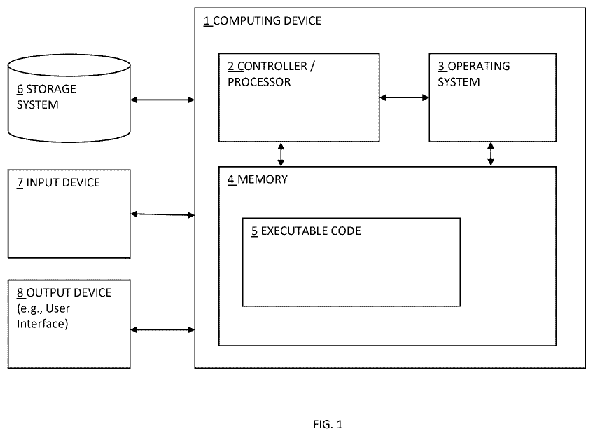

Reference is now made to FIG. 1, which is a block diagram depicting a computing device, which may be included within an embodiment of a system for determining a condition of a biological sample based on microscopy images, according to some embodiments.

Computing device 1 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task, or script. Executable code 5 may be executed by processor or controller 2 possibly under control of operating system 3. For example, executable code 5 may be an application that may analyze microscopy images as further described herein. Although, for the sake of clarity, a single item of executable code 5 is shown in FIG. 1, a system according to some embodiments of the invention may include a plurality of executable code segments similar to executable code 5 that may be loaded into memory 4 and cause processor 2 to carry out methods described herein.

Storage system 6 may be, or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data pertaining to sampled microscopy images may be stored in storage system 6 and may be loaded from storage system 6 into memory 4 where it may be processed by processor or controller 2. In some embodiments, some of the components shown in FIG. 1 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components, or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

The term neural network (NN) or artificial neural network (ANN), e.g., a neural network implementing a machine learning (ML) or artificial intelligence (AI) function, may be used herein to refer to an information processing paradigm that may include nodes, referred to as neurons, organized into layers, with links between the neurons. The links may transfer signals between neurons and may be associated with weights. A NN may be configured or trained for a specific task, e.g., pattern recognition or classification. Training a NN for the specific task may involve adjusting these weights based on examples. Each neuron of an intermediate or last layer may receive an input signal, e.g., a weighted sum of output signals from other neurons, and may process the input signal using a linear or nonlinear function (e.g., an activation function). The results of the input and intermediate layers may be transferred to other neurons, and the results of the output layer may be provided as the output of the NN. Typically, the neurons and links within a NN are represented by mathematical constructs, such as activation functions and matrices of data elements and weights. At least one processor (e.g., processor 2 of FIG. 1) such as one or more CPUs or graphics processing units (GPUs), or a dedicated hardware device may perform the relevant calculations.

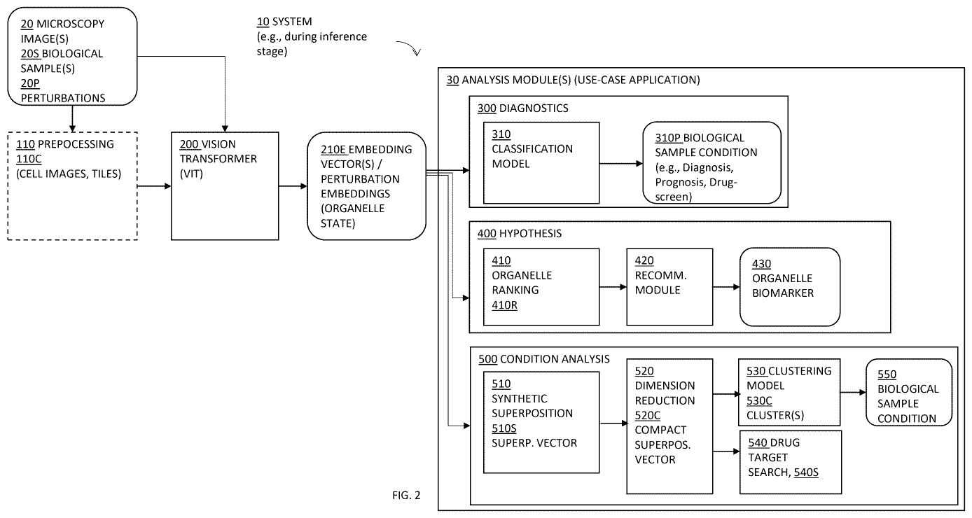

Reference is now made to FIG. 2, which depicts a system 10 for determining a condition of a biological sample 20S based on analysis of microscopy images 20, according to some embodiments.

According to some embodiments of the invention, system 10 may be implemented as a software module, a hardware module, or any combination thereof. For example, system may be or may include a computing device such as element 1 of FIG. 1, and may be adapted to execute one or more modules of executable code (e.g., element 5 of FIG. 1) to analyze microscopy images 20, as further described herein.

As shown in FIG. 2, arrows may represent flow of one or more data elements to and from system 10 and/or among modules or elements of system 10. Some arrows have been omitted on FIG. 2 for the purpose of clarity.

According to some embodiments, system 10 may receive one or more microscopy images 20 associated with a biological sample, where each microscopy image 20 depicts cellular organelles pertaining to specific organelle types.

As shown in FIG. 2, system 10 may include a preprocessing module 110 adapted to receive microscopy images 20 as input and perform preprocessing steps to filter and crop the microscopy images 20 into valid cell images 110C, depicting single cells, or tiles 110T which may depict more than a single cell. The terms cell image 110C and tile 110T may be used herein interchangeably, in this context.

According to some embodiments, microscopy images 20 may be treated with one or more specific stains or fluorescent labels to selectively highlight particular organelles within the cells. This selective staining technique may enhance the visibility and differentiation of specific organelle types. In some cases, multiple staining techniques may be applied simultaneously to visualize different organelles within the same sample 20S. For example, a biological sample may be treated with MitoTracker Green to specifically label mitochondria, LysoTracker Red to highlight lysosomes, and Hoechst 33342 to stain cell nuclei. Preprocessing module 110 may thereby produce cell images 110C that depict organelles pertaining to specific organelle types (e.g., mitochondria, nuclei, etc.) in cell image 110C. Cell images 110C or tiles 110T may be input to a vision transformer 200, for further analysis.

According to some embodiments, system 10 may infer vision transformer 200 on the microscopy images 20 (e.g., on cell images 110C or tiles 110T) to generate, for each organelle type, a corresponding embedding vector 210E, as explained herein.

Vision transformer 200 may be initially trained, or pre-trained on a first dataset of microscopy images 20 depicting non-perturbed (or null-perturbed) 20P biological samples 20S, to generate baseline embedding vectors 210E. These baseline embedding vectors 210E may encode states of organelles of respective types in the non-perturbed 20P biological samples.

The organelle states encoded in the baseline embedding vectors may include, for example localization patterns of respective organelles within the microscopy images, morphological patterns of the respective organelles, and properties of functionality of the respective organelles.

In some cases, the organelle states may include additional detailed characteristics such as size variations of the respective organelles, shape alterations of the respective organelles, distribution patterns of the respective organelles within cells, clustering or dispersion of the respective organelles, interactions between different types of organelles, membrane integrity of the respective organelles, protein composition of the respective organelles, lipid composition of the respective organelles, enzymatic activity within the respective organelles, oxidative stress markers in the respective organelles, organelle-specific protein aggregation, organelle-specific gene expression profiles, and post-translational modifications of organelle-associated proteins.

As explained herein, in a subsequent stage, vision transformer 200 may be trained using a contrastive learning framework, or approach, to distinguish between different perturbations 20P of the organelles of the biological samples 20S. Output embedding vectors 210E may then be referred to as perturbation embeddings 210E. It may be appreciated that the terms output embedding vectors 210E and perturbation embeddings 210E may be used interchangeably, to reflect an output of vision transformer 200, according to context.

As explained herein, system 10 may apply at least one analysis model 30 on embedding vectors (perturbation embeddings) 210E to predict the condition of the biological sample.

In some cases, analysis model(s) 30 may include a diagnostics module 300 which may be, or may include a machine-learning (ML) based classification model 310. Classification model 310 may be configured to, or trained to, produce a biological condition prediction 310P.

Prediction 310P of the biological sample may relate, for example, to a condition of disease of the underlying biological sample 20S. In such cases, prediction 310P may include a diagnosis of a disease state, a prognosis of a disease state, and the like.

In another example, predicted condition 310P may relate to a condition of a cellular response, and may include identification of such a response as a cellular stress response, a cellular drug response, a response to an environmental condition, and the like.

In another example, predicted condition 310P may relate to a condition of cellular functionality, and may include identification of a functional state such as a genetic mutation status, a developmental stage, a metabolic state, an activation state of a cellular pathway, a differentiation state of cells, an aging-related state, a cell cycle phase, an inflammatory state, a protein aggregation state, an organelle dysfunction state, and the like.

According to some embodiments, system 10 may include additional analysis models 30, such as hypothesis module 400. As shown in FIG. 2, hypothesis module 400 may include an organelle ranking module 410 adapted to generate an organelle rank or score 410R, from which system 10 may identify at least one organelle type as a biomarker 430, e.g., for drug development in treating a specific disease.

Additionally, or alternatively, system 10 may include additional analysis models 30 such as condition analysis module 500. As explained herein, module 500 may be configured to process perturbation embeddings 210E through components such as a synthetic superposition module 510 and a dimension reduction module 520 to produce compact representation vectors 520C of the organelles in biological samples.

In some embodiments, compact representations 520C may be clustered to a plurality of clusters 530C in a clustering model 530. Each cluster 530C may represent cells that have undergone, or manifest specific perturbations. System 10 may infer clustering model 530 on compact representation vectors 520C originating from target biological samples, to determine a condition of these target biological samples.

Additionally, and as elaborated herein, condition analysis module 500 may employ a drug target search algorithm 540, to identify at least one substance a target for drug development based on that substance's effect on compact representation vectors 520C in the vector space of clustering model 530.

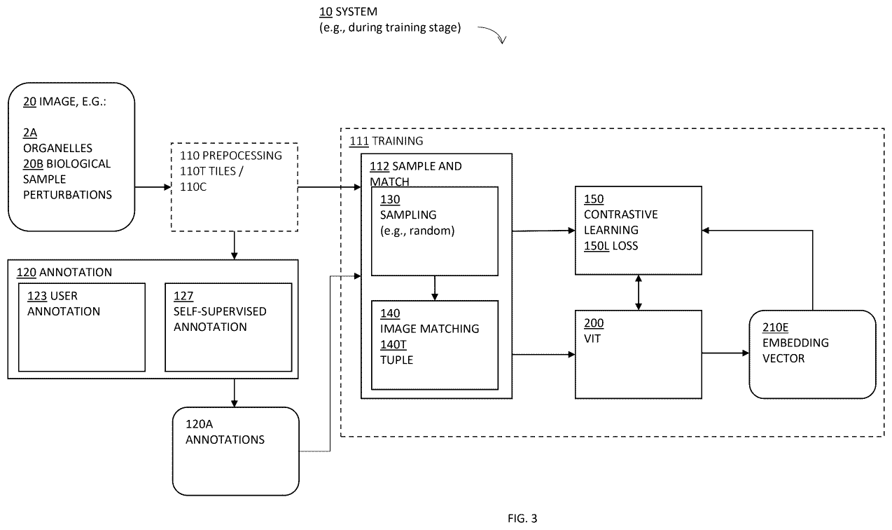

Reference is further made to FIG. 3, which is a block diagram showing operation of the system during a training stage, according to some embodiments of the invention.

According to some embodiments, system 10 may obtain, e.g., during a contrastive learning stage, a second dataset of microscopy images 20 or cell images 110C. Images 20/110C of the second dataset may depict organelles of a specific type, and may be extracted from perturbed 20P biological samples 20S. These microscopy images 20/110C may be annotated 120A according to the perturbation applied to, or manifested by, the biological samples 20S.

The annotation data 120A associated with images 20/110C may serve as supervisory data for retraining, or fine-tuning vision transformer 200, based on images 20/110C of the second dataset. System 10 may thereby generate an operational version of vision transformer 200. The term “operational” may be used in this context to indicate vision transformer's 200 capability to generate perturbation embeddings that associate states of organelles of specific types with respective biological sample perturbations 20P, as discussed herein.

Perturbations 20P used in this process may pertain to various types. For example, perturbations 20P may include null perturbations, disease-related perturbations, and experimental manipulations of the biological sample. Additionally, the perturbations may include genetic modifications, drug treatments, environmental stress conditions, metabolic alterations, cellular differentiation processes, and aging-related changes.

As shown in FIG. 3, system 10 may include an annotation module 120, adapted to generate annotation data 120A for corresponding microscopy images 20 (cell images 110C). Annotation data 120A may include information describing specific perturbations 20P that may have been applied to, or may be manifested in, corresponding biological samples 20S.

For example, annotation data 120A may indicate disease-related perturbations 20P, e.g., when the underlying biological sample has been extracted from a diseased tissue. In another example, annotation data 120A may indicate treatment-related perturbations 20P, e.g., when the biological sample has been administered with a specific substance or drug. In another example, annotation data 120A may indicate environment-related perturbations 20P, e.g., when the biological sample has been exposed to specific environmental condition such as heat, electric field, radiation, deprivation of oxygen, and the like. In another example, annotation data 120A may indicate a null perturbation 20P, e.g., when no manipulation, exposure or treatment has been applied to the biological sample, and/or when the biological sample is healthy (i.e., not diagnosed with a particular disease).

According to some embodiments, annotation module 120 may generate annotation data 120A as user annotations 123, e.g., by prompting an expert user (e.g., via output device 8 of FIG. 1) and receiving annotations 123 as input (e.g., via input device 7 of FIG. 1). Additionally, or alternatively, annotation module 120 may generate annotation data 120A as self-supervised annotations 127, as elaborated further herein.

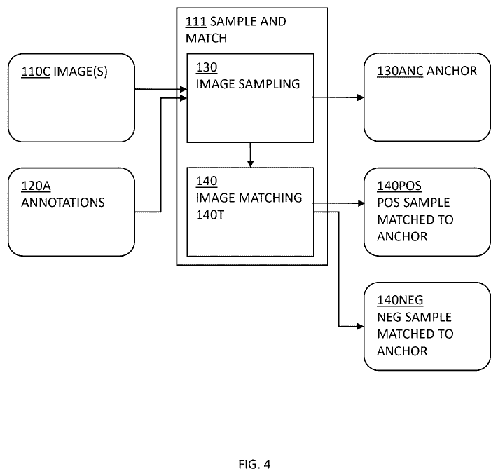

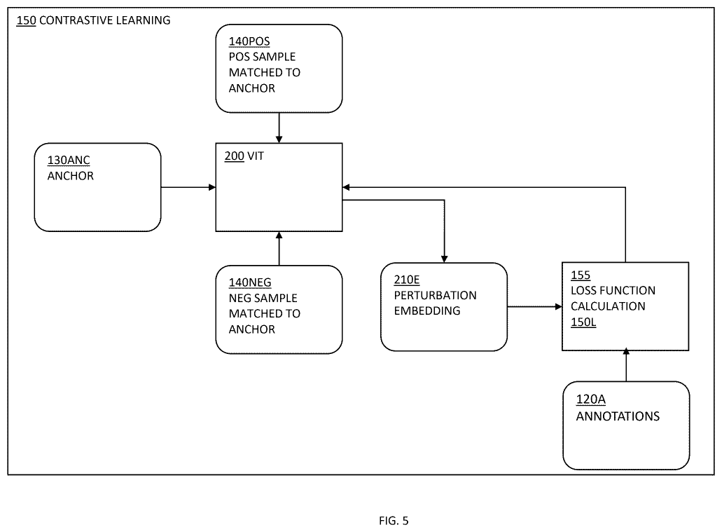

System 10 may include components for image matching and contrastive learning to train the vision transformer 200. Reference is now made to FIG. 4, and FIG. 5, which are block diagrams, that further elaborate these aspects. FIG. 4 depicts a sample and match module 111 that may be included in system 10 and may be adapted to produce a plurality of tuples 140T. FIG. 5 illustrates a contrastive learning dataflow, that may be employed by system 10 during a training stage, to train vision transformer 200 based on the selected tuples 140T, according to some embodiments of the invention.

According to some embodiments, sample and match module 111 may include an image sampling module 130. Image sampling module 130 may receive cell images 110C and annotation data 120A as input. Based on these inputs, the image sampling module 130 may select anchor images 130ANC in an iterative process: In each iteration, sampling module 130 may select an iteration-specific anchor image 130ANC. The anchor image 130ANC may be regarded as a representative image of a specific perturbation that is manifested by specific organelles of cell images 110C, as determined by the annotation data 120A.

System 10 may also include an image matching module 140, adapted to select, or compile one or more tuples 140T of microscopy images 110C of the second dataset. Each tuple's 140T microscopy images 110C may depict organelles of a specific type, originating from biological samples that pertain to two or more different perturbations.

According to some embodiments, each tuple 140T may include (i) an anchor image 130ANC depicting organelle(s) of a specific type pertaining to a first perturbation; (ii) a positive sample image 140POS depicting other organelle(s) of the same specific type, pertaining to the first perturbation; and (iii) a negative sample image 140NEG depicting another organelle of the same specific type pertaining to a second, different perturbation.

In other words, image matching module 140 may process the anchor image 130ANC to select or sample two types of additional cell images 110C: at least one positive sample 140POS, and at least one negative sample 140NEG. In some cases, the image matching module 140 may select one positive sample 140POS and five negative samples 140NEG for each anchor image 130ANC. The positive sample image(s) 140POS may represent samples matched to the anchor image 130ANC under similar perturbation conditions. The negative samples 140NEG may represent samples matched to the anchor image 130ANC under different perturbation conditions.

For example, anchor image 130ANC may be a cell image 110C depicting organelles (e.g., mitochondria) of liver tissue. Annotation data 120A may associate anchor image 130ANC with a specific perturbation, e.g., a disease-related perturbation. For example, annotation 120A may indicate that the biological sample of cell image 110C originates from a patient diagnosed with non-alcoholic fatty liver disease (NAFLD).

Anchor image 130ANC may be regarded as a representative image of this specific perturbation and organelle type, for that tuple 140T. In this case, anchor image 130ANC may represent NAFLD that is manifested by specific organelles, such as mitochondria. This manifestation may be determined by the annotation data 120A, which may include information such as tissue type, disease state, organelle of interest, and observed perturbation.

As known in the art, mitochondria characteristic of NAFLD may exhibit morphological changes, such as fragmentation, and reduced elongation, compared to mitochondria in healthy liver cells. Image matching module 140 may process the anchor image 130ANC to select or sample two types of additional cell images 110C: at least one positive sample 140POS, and at least one negative sample 140NEG. In selecting positive samples 140POS, the image matching module 140 may identify cell images 110C depicting mitochondria from other NAFLD-annotated biological samples, which may exhibit similar morphological changes as the anchor image 130ANC, such as fragmentation and reduced elongation compared to healthy liver cells. For negative samples 140NEG, the image matching module 140 may select cell images 110C depicting mitochondria from biological samples not annotated as NAFLD, such as those extracted from healthy tissue (null-perturbation) or liver tissue affected by a different disease. This selection process may enable image matching module 140 to create tuples 140T that effectively contrast NAFLD-specific mitochondrial changes with both healthy and other disease states.

In another example, the inventors have observed differences in morphology and localization of P-bodies between samples of amyotrophic lateral sclerosis (ALS) positive and negative tissue. In ALS-positive samples, P-bodies exhibit the following morphological changes compared to ALS-negative (control) samples. For example, P-bodies are generally smaller in ALS-positive samples. This is observed in both cytoplasmic TDP-43 positive C9ALS neurons and in human C9ALS post-mortem motor cortex tissue. In another example, while P-bodies are smaller, their number is increased in ALS-positive samples compared to controls. This is seen in C9ALS neurons and confirmed in human C9ALS post-mortem motor cortex tissue. In another example, ALS-positive samples, particularly in C9ALS patient neuropathology, show large intra-neuronal and extracellular LSM14A-positive bodies that are almost completely absent in control tissues. In yet another example. in cellular models with induced cytoplasmic TDP-43 (mimicking ALS conditions), P-bodies show reduced liquidity. The internal diffusion of P-body components (such as YFP-DCP1A) is delayed compared to controls. These morphological differences suggest that ALS-related perturbations, particularly those involving cytoplasmic TDP-43, significantly alter P-body dynamics and structure. The presence of smaller but more numerous P-bodies, along with the appearance of large LSM14A-positive bodies in ALS samples, indicates a substantial reorganization of these RNA-processing organelles in the disease state.

Pertaining to the ALS example, image matching module 140 may process an anchor image 130ANC depicting P-bodies in ALS-positive tissue to select corresponding positive 140POS and negative 140NEG samples. For the anchor image 130ANC, the module may choose a cell image 110C showing smaller, more numerous P-bodies with altered distribution, including large intra-neuronal and extracellular LSM14A-positive bodies, characteristic of ALS pathology. When selecting positive samples 140POS, the image matching module 140 may identify other ALS-positive cell images 110C exhibiting similar P-body alterations, such as reduced size, increased number, and the presence of large LSM14A-positive bodies. For negative samples 140NEG, the module may select cell images 110C from ALS-negative tissue, showing P-bodies with normal size, distribution, and absence of large LSM14A-positive bodies. Additionally, the image matching module 140 may consider the biophysical properties of P-bodies, selecting positive samples 140POS that demonstrate reduced liquidity and delayed internal diffusion of P-body components, and negative samples 140NEG with normal P-body dynamics. This selection process may enable the creation of tuples 140T that effectively contrast ALS-specific P-body changes with healthy states, facilitating the learning of disease-related perturbations in the vision transformer 200.

As shown in FIG. 5, for each tuple 140T, vision transformer 200 may process anchor image 130ANC, positive sample 140POS, and negative sample 140NEG, to generate perturbation embeddings 210E. The perturbation embeddings 210E may represent learned features from the processed images.

System 10 may include a loss function calculator 155. During contrastive-loss learning, vision transformer 200 may produce interim perturbation embeddings 210E. Loss function calculator 155 may receive the interim values of perturbation embeddings 210E, as well as annotations 120A. The loss function calculator 155 may compute a contrastive loss function 150L based on this input, as a measure of difference between the perturbation embeddings 210E.

The contrastive learning module 150 may use the contrastive loss function 150L to guide an iterative training process for the vision transformer 200. In each iteration, the loss function calculator 155 may compute a value for the contrastive loss function 150L pertaining to a specific tuple of images.

The contrastive loss function 150L may be calculated such as to (i) minimize variation between perturbation embeddings 210E that pertain to similar perturbations, e.g., between anchor image 130ANC and positive image samples 140POS, and (ii) accentuate variation between perturbation embeddings 210E that pertain to different perturbations, e.g., between anchor image 130ANC and negative image samples 140POS.

System 10 may thus fine-tune vision transformer 200 using the second dataset of microscopy images, thereby generating the operational version of the vision transformer 200, which is adapted to produce perturbation embeddings 210E. As explained herein, perturbation embeddings 210E may associate states of organelles of specific types with respective biological sample perturbations.

System 10 may update parameters of the vision transformer 200 by backpropagating gradients computed from the contrastive loss function 150L. This process may allow the vision transformer 200 to learn to distinguish between different perturbations of the organelles depicted in the microscopy images 20, while generalizing manifestation of similar perturbations in images 110C.

Pertaining to the ALS example provided above, vision transformer 200 may initially generate similar embeddings for all three images 130ANC, 140POS and 140NEG. As training progresses, the updating of parameters may cause: (a) the embeddings for anchor image 130ANC and positive sample 140POS to become more similar, reflecting their shared ALS-related P-body changes, such as reduced size, increased number, and the presence of large LSM14A-positive bodies; (b) The embedding for negative sample 140NEG to become more distinct from the other two, reflecting the difference between healthy and ALS-affected P-bodies. This process may be repeated iteratively, across many tuples 140T, allowing vision transformer 200 to learn to distinguish between various perturbations (e.g., ALS vs. healthy vs. other neurodegenerative diseases) while generalizing across similar perturbations (e.g., different cases of ALS with slightly varying P-body presentations).

Similarly, pertaining to the example of NAFLD provided above, the updating of parameters may cause: (a) the embeddings for anchor image 130ANC and positive sample 140POS to become more similar, reflecting their shared NAFLD-related mitochondrial changes; (b) The embedding for negative sample 140NEG to become more distinct from the other two, reflecting the difference between healthy and NAFLD-affected mitochondria. This process may be repeated across many tuples 140T, allowing vision transformer 200 to learn to distinguish between various perturbations (e.g., NAFLD vs. healthy vs. other liver diseases) while generalizing across similar perturbations (e.g., different cases of NAFLD with slightly varying presentations).

In an experimental implementation, vision transformer 200 was trained on a corpus of approximately 3.2 million microscopy images of human iPSC-derived neurons acquired under multiple experimental conditions, e.g., wild-type neurons, chemical perturbations, and genetic perturbations. The corpus was partitioned into about 2.24 million images for model training, about 0.48 million images for validation, and about 0.48 million images for testing. The training dataset was assembled from two independent biological differentiations (experimental repeats), each including two or eight technical repeats per condition.

To better capture non-linear relationships in the data, ViT 200 may include a multi-layer head. Additionally, contrastive learning may be configured across experimental repeats, and attention maps may be generated during training and/or inference to support interpretability and quality control. As used herein, and consistent with Materials Design Analysis Reporting (MDAR) guidelines, an “experimental repeat” may refer to an independent biological differentiation of a cell line or a human subject.

As shown in the implementation example of FIG. 2, system 10 may include one or more (e.g., three) analysis paths, or modules for processing the perturbation embeddings 210E generated by the vision transformer 200. These include a diagnostics module 300, a hypothesis module 400, and a biological condition analysis module 500. Each module may process the perturbation embeddings 210E to generate different types of insights about the biological samples depicted in the microscopy images 20.

In some cases, the diagnostics module 300 may include a classification model 310. The classification model 310 may be a Machine-Learning (ML) based model that may be trained to predict the biological sample condition from the perturbation embeddings 210E.

During a training stage, the classification model 310 may be trained to produce prediction 310P of the sample's condition based on annotations 120A. The training process may involve feeding the model with a large dataset of perturbation embeddings 210E generated by the vision transformer 200, along with their corresponding annotations 120A. These annotations 120A may include information about the biological conditions associated with each embedding, such as disease states, cellular responses, or functional states. Classification model 310 may use supervised learning techniques, such as gradient descent optimization, to adjust its internal parameters and learn the relationships between the perturbation embeddings 210E and the annotated conditions. This iterative process may continue until the model achieves a satisfactory level of accuracy in predicting the biological conditions from the training data.

During a subsequent inference stage, predictions 310P for new cell images 110C may be obtained by first processing these images through the trained vision transformer 200 to generate their corresponding perturbation embeddings 210E. These embeddings may then be input into the trained classification model 310, which may apply the learned relationships to produce predictions 310P of the biological conditions associated with the new images. The classification model 310 may output these predictions 310P as probabilities or confidence scores for various possible conditions, allowing for a nuanced interpretation of the results. In some cases, diagnostics model 300 may also provide additional information, such as the key features or organelle types that contributed most significantly to the prediction 310P, offering insights and interpretability into the underlying biological mechanisms.

Hypothesis module 400 may include an organelle ranking module 410. The organelle ranking module 410 may quantify and rank organelle types based on their discriminatory power between perturbations. In some cases, the organelle ranking module 410 may use a predetermined delta metric to quantify the effect of perturbation on specific organelles. Organelle ranking module 410 may subsequently generate an organelle score 410R, representing this discriminatory power for one or more (e.g., each) organelle type.

In some embodiments, the organelle ranking module 410 may apply a mixed-effects meta-analytic model to estimate a combined effect size for each organelle type under a given perturbation. This approach may account for sampling variance within experimental repeats (uncertainty) and heterogeneity across experimental repeats (random effects), thereby enabling statistical assessment of whether an organelle is consistently affected (reproducibility) across biological differentiations and/or human subjects.

In certain implementations, for each perturbation examined (e.g., during inference), perturbation embeddings 210E derived from control images may be compared to perturbation embeddings 210E derived from perturbed images to estimate an effect size per organelle per experimental repeat. The effect size may be regarded as a quantification of the degree to which a perturbation shifts organelle topography relative to a baseline state and may be computed for each combination of perturbation, organelle type, and experimental repeat, together with an estimate of sampling variance to reflect repetition uncertainty.

According to some embodiments, repeat-wise effect sizes may be combined using a mixed-effects meta-analytic model to obtain a combined effect size for each organelle type to capture heterogeneity across repeats. This framework may yield a standard error and confidence interval for the combined effect size and may permit hypothesis testing as to whether an organelle is consistently affected. Multiple-hypothesis correction may be applied across organelle types and/or perturbations.

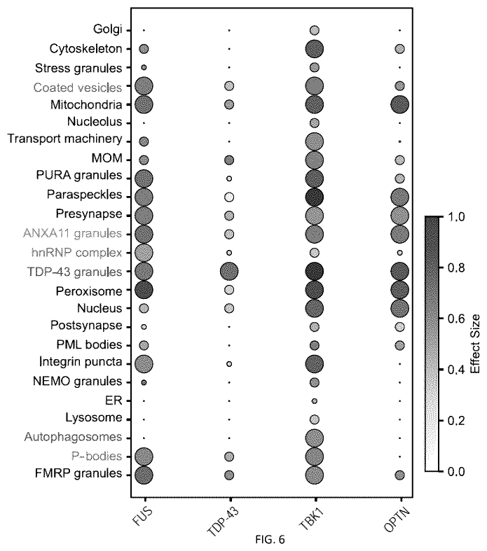

Reference is now made to FIG. 6, which is a bubble diagram, showing experimental results, by which organelle scores 410R were calculated for different organelle types (Y axis, e.g., Golgi, Cytoskeleton, Stress granules, etc.), in relation to different types of perturbations (X axis, e.g., Fused in Sarcoma(FUS), (TAR DNA/RNA-Binding Protein 43 (TDP-43), TANK Binding Kinase 1 (TBK1 ), and Optineurin(OPTN)). In the example of FIG. 6, organelle score 410R is a dual-value vector: The effect of perturbations on the perturbation embeddings 210E is presented by the respective bubble's color, whereas the statistical significance of this effect is demonstrated by each bubble's size.

In the example of FIG. 6, organelle ranking module 410 may use a predetermined delta metric such as the Cliff delta metric, to quantify the effect of perturbation on specific organelle types. The delta metric may range from −1 to 1, where a value of 1 may indicate that all values in one group are higher than all values in the other group, a value of −1 may indicate that all values in one group are lower than all values in the other group, and a value of 0 may indicate complete overlap between the two groups.

Additionally, or alternatively, the delta metric may be implemented as a distance-based log2 fold-change effect size combined with a random-effects meta-analysis. In some embodiments, per-repeat distances between control and perturbed embeddings (e.g., centroid Euclidean distance or median pairwise distance) may be expressed as a log2 fold-change relative to a baseline dispersion. The repeat-wise log2 effects and sampling variances may then be pooled using a random-effects model to obtain a combined effect size with confidence interval and adjusted significance. Organelle scores 410R may be derived from the magnitude and/or adjusted significance of this combined log2 effect.

Organelle ranking module 410 may subsequently generate organelle score 410R for one or more (e.g., each) organelle type, to indicate (i) amplitude of a specific perturbation's effect on that organelle, and (ii) statistical significance of that effect, as presented in FIG. 6.

Additionally, or alternatively, organelle scoring may be derived from a mixed-effects meta-analytic analysis of repeat-wise effect sizes computed from embeddings vectors 210E. In some embodiments, effects may be presented in organelle scoring forest plots that display per-repeat effect sizes with uncertainty alongside the combined effect size and confidence interval. Organelle rankings (e.g., 410R) may then be based on the magnitude and/or adjusted statistical significance of the combined effect size.

As shown in FIG. 2, hypothesis module 400 may also include a recommendation module 420, configured to identify and analyze a subset of highly ranked organelle types based on the organelle scores 410R. For example, recommendation module 420 may identify organelle scores 410R that (i) have a high effect, e.g., beyond a first predetermined threshold on perturbation embeddings 210E, while (ii) demonstrating statistical significance beyond a second predefined threshold. Recommendation module 420 may thereby determine the relevance of organelle types of the identified subset to a specific perturbation (e.g., a specific disease), or condition of the underlying biological samples, as indicated by ranking 410R.

In some embodiments, the identified subset may be prioritized using thresholds defined on the meta-analytic combined effect size and on adjusted significance values derived from the mixed-effects model, thereby emphasizing organelles exhibiting large, reproducible responses across experimental repeats.

Recommendation module 420 may subsequently designate one or more organelle types from the identified subset as an organelle biomarker 430 for drug development. Such drug development process may include several stages. Initially, researchers may conduct high-throughput screening of chemical compounds to identify those that affect the designated organelle biomarker 430 in a desired manner. Promising compounds may then undergo optimization to enhance their efficacy and reduce potential side effects. Subsequently, preclinical studies may be performed to assess the safety and efficacy of the optimized compounds in cellular and animal models. Successful candidates may progress to clinical trials, where their effects on human subjects are evaluated in phases, starting with safety assessments in healthy volunteers and advancing to efficacy studies in patients with the target condition. Throughout this process, the impact of the drug candidates on the organelle biomarker 430 may be monitored to guide decision-making and refine the drug's mechanism of action.

### Biological Condition Analysis Module 500

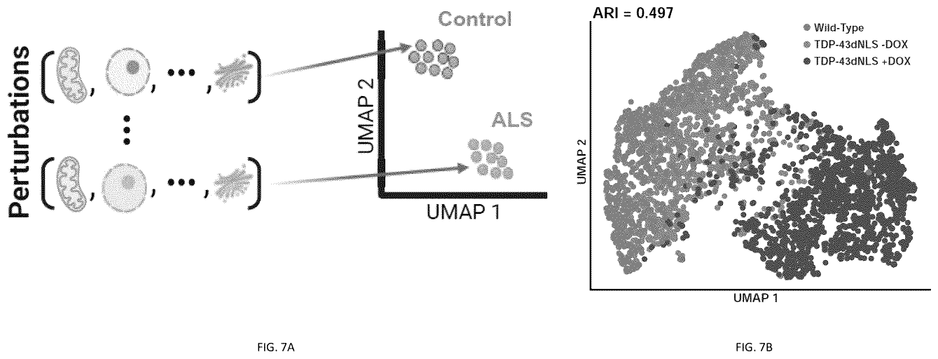

Reference is further made to FIG. 7A, which is a schematic diagram showing the workflow of the biological condition analysis module 500, and to FIG. 7B, which is a scatter graph, showing experimental results for predicting a condition of ALS by biological condition analysis module 500, according to some embodiments of the invention.

As shown in FIG. 2, biological condition analysis module 500 may include a synthetic superposition module 510. Synthetic superposition module 510 may combine perturbation embeddings 210E that pertain to different organelle types, but represent a common perturbation, to generate a superposition vector 510S. In their implementation, the inventors have combined perturbation embeddings 210E from as many as 25 different organelles, to produce synthetic superposition vectors 510S.

In some embodiments, condition analysis 500 may explicitly study the sizes and distances of perturbations in the full embedding space with a Euclidean distance function. Additionally, or alternatively, condition analysis 500 may analyze a reduced-dimension representation of the perturbation embedding space.

The biological condition analysis module 500 may include a dimension reduction module 520, configured to perform dimensionality reduction on the superposition vector 510S to generate a compact superposition vector 520C, representing an integrated organellome view (i.e., pertaining to a variety of organelle types) of a specific biological sample perturbation. In some cases, the dimensionality reduction may be performed using Uniform Manifold Approximation and Projection (UMAP).

Biological condition analysis module 500 may further include a biological condition clustering model 530 (or “clustering model 530”, for short). Clustering model 530 may be configured to obtain a plurality of compact superposition vectors 520C, pertaining to a respective plurality of biological samples, and group compact superposition vectors 520C into clusters, based on a predetermined distance metric such as a Euclidean distance. Each cluster of clustering model 530 may represent a specific perturbation 20P of respective biological samples 20S.

This process is described schematically in FIG. 7A. For each subject of a group of control (healthy) subjects, a first (cyan) set of perturbation embeddings 210E are extracted, each representing a respective organelle type (whole cells, Nuclei, Golgi, etc.). The perturbation embeddings 210E are aggregated into a first superposition vector 510S, from which a compact superposition vector 520C (e.g., UMAP) is produced. Each point in the cluster of cyan “control” points represents a compact superposition vector 520C of a respective subject of the control group.

In a complementary manner, for each subject of a group of ALS-positive subjects, a second (orange) set of perturbation embeddings 210E are extracted, representing the respective organelle types. The perturbation embeddings 210E are aggregated into a second superposition vector 510S, from which a compact superposition vector 520C (e.g., UMAP) is produced. Each point in the cluster of orange “ALS” points represents a compact superposition vector 520C of a respective subject of the group of ALS-positive subjects.

FIG. 7B is a scatter plot, depicting an example of a clustering model 530 which may be obtained by embodiments of the invention, in the context of analyzing disease-related (ALS) perturbations.

The scatter plot of FIG. 7B represents different experimental conditions related to TDP-43 protein and their effect on cellular processes, which may be relevant to the study of ALS. Each color of the plot corresponds to a specific experimental condition and/or cohort of subjects.

Cyan dots represent a “wild type” condition. This condition may refer to cells expressing a normal, unmodified version of TDP-43 protein with an intact Nuclear Localization Signal (NLS), allowing the TDP-43 protein to properly localize to the nucleus under normal conditions. These dots may serve as a baseline or control, potentially showing the natural variation in cellular characteristics or organelle behavior in healthy cells.

Green dots represent a “TDP-43dNLS-DOX” condition. This condition may involve cells expressing a modified TDP-43 protein, lacking its nuclear localization signal (dNLS), without doxycycline (DOX) induction. These cells may exhibit some alterations compared to the wild type, due to the presence of the modified protein, but the effects may be less pronounced than in the doxycycline-induced condition.

Purple dots represent a “TDP-43dNLS +DOX” condition. This condition may involve cells expressing the modified TDP-43 protein (dNLS) and induced with doxycycline (DOX). The doxycycline induction may result in higher expression or accumulation of cytoplasmic TDP-43, potentially mimicking the pathological condition observed in ALS.

The distribution and clustering of these dots may provide several insights. For example, the separation of clusters may suggest that each condition results in a unique cellular state or phenotype. The degree of separation between clusters may indicate the extent of differences between the conditions. Any overlap between clusters, particularly between TDP-43dNLS-DOX and +DOX, may indicate a gradual change in cellular state as TDP-43 accumulates in the cytoplasm. The distance of the TDP-43dNLS clusters (both −DOX and +DOX) from the wild type cluster may indicate the degree of cellular changes caused by the modified protein. The spread of dots within each cluster may represent the variability of cellular responses within each condition.

The results presented in this plot may be used to quantify the effects of cytoplasmic TDP-43 accumulation on cellular processes, potentially including changes in organelle behavior, gene expression patterns, or other cellular characteristics relevant to ALS pathology. A clear separation of the TDP-43dNLS +DOX condition from the others may suggest that induced cytoplasmic accumulation of TDP-43 causes significant and consistent changes in cellular state, which may support its role in ALS pathogenesis.

As shown in the example of FIG. 7B, clustering of compact superposition vectors 520C, which encapsulate a comprehensive organellome view in a reduced dimensionality, may reveal distinct groupings that correspond to unique cellular states or phenotypes associated with different conditions. This approach may enable researchers to visualize and analyze complex cellular changes across multiple organelles simultaneously, potentially uncovering subtle, yet significant alterations in cellular physiology that may not be apparent when examining individual organelles in isolation. By leveraging this holistic representation of the organellome, the clustering method may therefore facilitate the identification of disease-specific signatures, and clarify mechanisms of pathogenesis, to potentially guide development of targeted therapeutic interventions.

Additionally, or alternatively, system 10 may utilize clustering model 530 to identify a condition 550 of a specific biological sample of interest. For example, biological condition analysis module 500 may calculate a predetermined distance metric value (e.g., Euclidean distance) between an incoming, target compact superposition vector 520C, originating from the biological sample of interest 20S, and one or more (e.g., each) cluster of clustering model 530. Biological condition analysis module 500 may subsequently associate the target compact superposition vector 520C with a specific cluster 530C of clustering model 530, to determine, or predict condition 550 of the biological sample of interest 20S.

Clusters 530C may be generated through an automated process that considers the characteristics of the subject cohort and various applied perturbations. As a result, the conditions 550 associated with these clusters 530C may not necessarily correspond to conventionally defined medical conditions. Instead, they may represent distinct cellular states or phenotypes that emerge from the data.

For example, based on the description of FIG. 7B, conditions 550 might include: (i) A condition associated with wild-type TDP-43 localization and function, represented by the cyan cluster; (ii) A condition characterized by the presence of modified TDP-43 lacking the nuclear localization signal (TDP-43dNLS), but without doxycycline induction, represented by the green cluster; and (iii) A condition marked by high cytoplasmic accumulation of TDP-43dNLS induced by doxycycline, represented by the purple cluster.

These conditions 550 may not directly align with traditional medical definitions of ALS or other neurodegenerative diseases. Instead, they may represent specific cellular states associated with TDP-43 dysfunction, which could be relevant to understanding the progression or subtypes of ALS. The automated clustering approach may reveal these distinct cellular states or conditions 550 without relying on pre-existing disease classifications, potentially uncovering novel insights into the spectrum of cellular responses to TDP-43 perturbations 20P.

The biological condition analysis module 500 may also include a drug target selector 540. According to some embodiments, drug target selector 540 may be configured to identify treatment-related perturbations that reverse disease-related perturbations in the vector space of the biological condition clustering model 530. In such embodiments, condition analysis module 500 may calculate a vector in the vector space of clustering model 530 that represents the disease-related perturbation, e.g., originating from a cluster associated with a healthy state and terminating at a cluster associated with a diseased state. Condition analysis module 500 may then calculate an opposite vector, which may have the same magnitude but opposite direction to the disease-related perturbation vector. This opposite vector may represent a trajectory for potentially reversing the disease state towards a healthy state.

Condition analysis module 500 may analyze various treatment-related perturbations by calculating vectors of change that these perturbations may induce in the vector space of clustering model 530. The drug target selector 540 may identify a treatment-related perturbation whose vector of change substantially matches the direction and amplitude of the opposite vector. This matching may be determined based on a predetermined similarity threshold. In some embodiments, one or more substances associated with the identified treatment-related perturbation may be designated for drug development to treat the specific disease represented by the disease-related perturbation. This approach may allow for the identification of potential drug targets based on their ability to induce cellular changes that may counteract disease-related alterations across multiple organelles, as represented in the space of compact superposition vector 520.

In some embodiments, the outputs from the analysis modules may be stored in the memory 4 or storage system 6 of the computing device 1 for further processing or visualization. Additionally, or alternatively, system 10 may be configured to present or visualize various outcomes on a User Interface (UI), such as output device 8 of computing device 1.

UI 8 may display, for example biological condition predictions 310P generated by the classification model 310, which may include, for example diagnoses, prognoses, or identified cellular responses pertaining to specific biological samples of interest 20S.

In another example, UI 8 may display organelle scores 410R produced by the organelle ranking module 410, potentially presented as a ranked list or heat map showing the discriminatory power of different organelle types in relation to specified perturbations 20P. Additionally, or alternatively, UI 8 may display identified organelle biomarkers 430, which may be displayed as highlighted organelle types or as a list of potential drug development organelle targets.

According to some embodiments, UI 8 may present organelle scoring forest plots, combined effect sizes with confidence intervals, heterogeneity summaries across experimental repeats, and multiple-hypothesis-adjusted significance values. Ranked lists may be annotated with the meta-analytic combined effect size and adjusted significance to facilitate interpretation and decision-making.

In another example, UI 8 may visualize clusters 530C of biological condition clustering model 530, such as scatter plots or dimensionality-reduced representations of the compact superposition vectors 520C, with different clusters color-coded to represent various biological conditions or perturbations. UI 8 may do so as background for displaying results from the drug target selector 540, highlighting effect of specific treatment on reversal of disease-related perturbations. UI 8 may also display a list of identified substances for drug development, along with visualizations of how these substances may reverse disease-related perturbations in the vector space.

In another embodiment, UI 8 may present interactive plots, allowing users to explore relationships between different organelle types, perturbations, and biological conditions. Additionally, or alternatively, UI 8 may display time-series visualizations, showing how cellular states may change over time or in response to different stages of treatment or perturbations 20P. Other such visualizations and analyses are also possible.

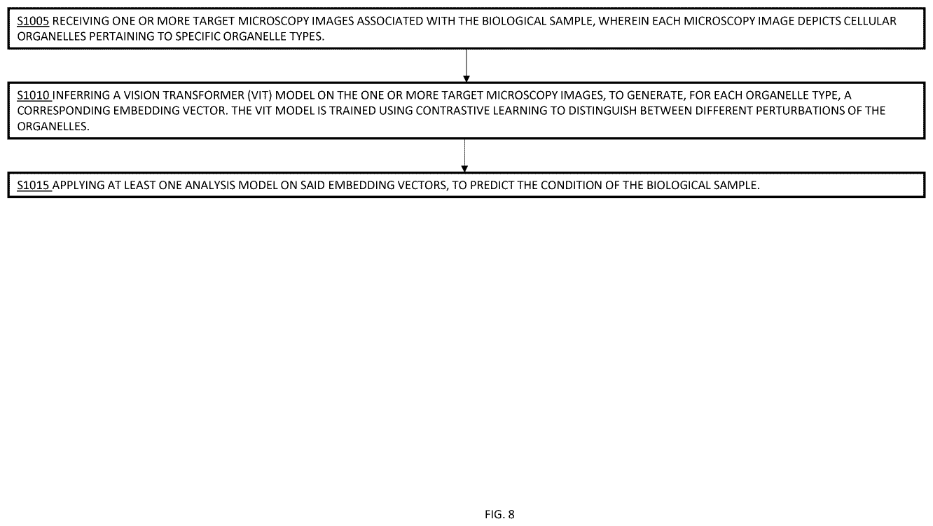

FIG. 8 is a flow diagram depicting a method of determining a condition of a biological sample by at least one processor (e.g., processor 2 of FIG. 1).

As shown in step S1005, the at least one processor 2 may receive one or more target microscopy images (e.g., images 20 of FIG. 2) associated with the biological sample. Each microscopy image 20 may depict cellular organelles pertaining to specific organelle types.

At step S1010, the at least one processor 2 may infer, or apply a Vision Transformer (ViT) model (e.g., ViT 200 of FIG. 2) on the one or more target microscopy images 20, to generate, for each organelle type, a corresponding embedding vector (e.g., 210 of FIG. 2). As explained herein, the ViT model 200 may be trained to produce embedding vectors 210 using contrastive learning, to distinguish between different perturbations of the organelles.

At step S1015, the at least one processor 2 may apply at least one analysis model (e.g., 300, 400, 500 in analysis 30 of FIG. 2) on the embedding vectors, to predict the condition (e.g., 310P, 430, 550 of FIG. 2), pertaining to the biological sample.

In some embodiments, the present invention provides a practical application, manifested by improvement of technology for biological sample analysis and computer-assisted diagnostics. By transforming multi-channel microscopy images into perturbation embeddings using the vision transformer 200 and organizing those embeddings into compact superposition vectors 520C, the system 10 may deliver enhanced diagnostic performance and decision support. This pipeline may reduce noise and inter-sample variability through contrastive pre-training and fine-tuning, and may integrate information across multiple organelle types via the synthetic superposition module 510. As a result, analysis model(s) 30 may detect disease-related perturbations and cellular states with improved sensitivity and robustness, and the biological condition predictions 310P may be produced more rapidly and consistently than conventional manual inspection workflows.

In some cases, these improvements may manifest in tangible benefits to laboratory and clinical workflows. The preprocessing module 110 may reduce image artifacts and standardize inputs at scale; the vision transformer 200 may generate embeddings 210E that are more separable across conditions, thereby decreasing downstream classification complexity; and the dimension reduction module 520 may compress high-dimensional organellome representations into compact superposition vectors 520C that are suited for fast clustering and retrieval. These computational enhancements may reduce turnaround time, increase throughput, and enable consistent, reproducible determinations across large cohorts, facilitating applications such as stratification of patient samples, monitoring of treatment effects, and identification of organelle biomarkers 430 for drug development.

In some embodiments, the system 10 may improve the functioning of the underlying computing device 1 by structuring image analysis as a sequence of specialized transformations that are compatible with parallel hardware execution. For example, batching tiles 110T and cell images 110C, applying patch-wise attention in the vision transformer 200, and computing contrastive loss 150L using vectorized distance operations may reduce memory transfers and increase cache locality. The generation of compact superposition vectors 520C may lower storage and bandwidth requirements for downstream clustering and retrieval in the biological condition clustering model 530, enabling near-real-time association of new samples with existing clusters 530C. These architectural and representational choices may yield lower latency and higher throughput compared to generic image analysis pipelines.

In some cases, practical application is further demonstrated through actionable outputs on the user interface 8, including biological condition predictions 310P, organelle scores 410R, recommended organelle biomarkers 430, and visualization of condition clusters 530C. The drug target selector 540 may identify treatment-related perturbations that reverse disease-related perturbations in the vector space of clustering model 530, supporting hypothesis generation and prioritization of substances for drug development. Collectively, these capabilities may enhance diagnostic confidence, reduce manual review burden, and support evidence-based therapeutic decision-making.

The methods explained herein are not amenable to performance by mental processes. The pipeline may operate on large, multi-channel microscopy datasets in which each image can comprise millions of pixels and multiple spectral channels. The vision transformer 200 may include millions of parameters, with training and fine-tuning requiring backpropagation of gradients through deep attention layers and optimization of high-dimensional weight tensors, which is not feasible without specialized processors and numeric libraries. Contrastive learning with tuples 140T requires computing pairwise or batch-wise distances between numerous embeddings 210E and aggregating loss values 150L across large batches, again necessitating vectorized linear algebra operations.

Additionally, the construction of compact superposition vectors 520C may involve aggregating embeddings across many organelle types and applying nonlinear manifold learning (e.g., UMAP) that builds approximate nearest-neighbor graphs and optimizes low-dimensional embeddings using iterative numerical procedures. Clustering model 530 may further require computing distances among large sets of compact vectors, updating cluster assignments, and evaluating stability across parameter sweeps. The drug target selector 540 may compute disease-related perturbation vectors, opposite vectors, and treatment-induced vectors of change, and then evaluate directional and magnitude similarity under a predetermined threshold across many candidate substances.

These calculations involve high-dimensional vector arithmetic, graph construction, and iterative optimization that cannot be executed mentally and may rely on floating-point operations, GPU acceleration, and substantial memory resources.

In some embodiments, the framework presented herein may provide a microscopy-based approach for large-scale interrogation of organellar cell biology that builds on standard immunofluorescence workflows. The system may enable quantitative, system-level comparisons of cellular responses to diverse perturbations, moving beyond compartment-specific analyses. The approach of the present invention may be compatible with multiple imaging platforms and may extend to patient-derived cell types and legacy imaging datasets, positioning organellome-based analysis as a broadly applicable discovery engine with translational potential e.g., in neurological disease research and precision medicine.

In certain implementations, high-plex imaging platforms (e.g., PhenoCycler, 4i) may require complex protocols, long acquisition times, and dedicated instrumentation. By contrast, organellome-based analysis as described herein may offer a cost-effective, scalable, and widely accessible alternative based on standard confocal imaging, thereby lowering barriers to adoption and increasing throughput.

Additionally, while many existing approaches may require prospective experimental design and fixed marker panels, embodiments of the invention may be applied retrospectively to existing microscopy datasets and may be agnostic to the number of fluorescent channels. This capability may enable immediate reuse of legacy data and flexible experimental expansion without redesigning marker panels.

Additionally, feature-engineering workflows (e.g., Cell Painting or CellProfiler-based profiling) may rely on predefined objects and standardized dye sets, yielding interpretable but constrained morphological summaries. By contrast, Vision Transformer 200 may employ deep representation learning to capture multi-scale organellar organization directly from endogenous marker distributions, avoiding explicit segmentation and generalizing more robustly across markers, perturbations, and conditions.

Embodiments of the invention may be effective e.g., across diverse brain cell types and may exhibit particular advantages in highly elaborated and polarized neurons, where other methods may fail or require extensive manual tuning. This robustness may improve sensitivity to disease-relevant organellar reorganization and reduce analyst intervention.

Additionally, or alternatively, the approach presented by embodiments of the invention may generalize to any marker and staining technology (e.g., antibodies, dyes), and may extend to large multiplex settings (e.g., greater than 100 markers). By operating on fixed-cell images, the framework presented herein may overcome scalability, phototoxicity, and multiplexing limitations inherent to live-cell imaging while retaining sensitivity to disease-relevant organellar changes. Collectively, these aspects may provide practical improvements in accessibility, scalability, and reproducibility for biological sample analysis and computer-assisted diagnostics.

1. A method of determining a condition of a biological sample by at least one processor, the method comprising:
receiving one or more target microscopy images associated with the biological sample, wherein each microscopy image depicts cellular organelles pertaining to specific organelle types;
inferring a Vision Transformer (ViT) model on the one or more target microscopy images, to generate, for each organelle type, a corresponding embedding vector, wherein the ViT model is trained using contrastive learning to distinguish between different perturbations of the organelles; and
applying at least one analysis model on said embedding vectors, to predict the condition of the biological sample.

2. The method of claim 1, wherein the predicted condition of the biological sample is selected from a list consisting of: a diagnosis of a disease state, a prognosis of a disease state, a cellular stress response, a drug response, a genetic mutation status, a developmental stage, a metabolic state, an activation state of a cellular pathway, a differentiation state of cells, an aging-related state, a cell cycle phase, an inflammatory state, a protein aggregation state, an organelle dysfunction state.

3. The method of claim 1, further comprising pre-training the ViT model on a first dataset of microscopy images, depicting non-perturbed biological samples, to generate baseline embedding vectors, encoding states of organelles of respective types in said non-perturbed biological samples.

4. The method of claim 3, wherein said organelle states are selected from a list consisting of localization patterns of respective organelles within the microscopy images, morphological patterns of the respective organelles, properties of functionality of the respective organelles, and any combination thereof.

5. The method of claim 3, wherein said organelle states are selected from a list consisting of: localization patterns of the respective organelles within the microscopy images, morphological patterns of the respective organelles, size variations of the respective organelles, shape alterations of the respective organelles, distribution patterns of the respective organelles within cells, clustering or dispersion of the respective organelles, interactions between different types of organelles, membrane integrity of the respective organelles, protein composition of the respective organelles, lipid composition of the respective organelles, enzymatic activity within the respective organelles, oxidative stress markers in the respective organelles, organelle-specific protein aggregation, organelle-specific gene expression profiles, post-translational modifications of organelle-associated proteins, and any combination thereof.

6. The method of claim 1, further comprising:
obtaining a second dataset of microscopy images, each (a) depicting organelles of a specific type, extracted from a perturbed biological sample, and (b) annotated according to said perturbation;
using the image annotations as supervisory data, to fine-tune the ViT model based on microscopy images of the second dataset, thereby generating an operational version of the ViT model, wherein said operational version is configured to generate perturbation embeddings that associate states of organelles of specific types with respective biological sample perturbations.

7. The method of claim 6, wherein said perturbations comprise null perturbations, disease-related perturbations and experimental manipulations of the biological sample.

8. The method of claim 6, wherein said perturbations comprise null perturbations, disease-related perturbations, genetic modifications, drug treatments, environmental stress conditions, metabolic alterations, cellular differentiation processes, and aging-related changes.

9. The method of claim 6, wherein fine-tuning the ViT model comprises:
selecting one or more tuples of microscopy images of the second dataset, wherein each tuple's microscopy images depict organelles of a specific type, originating from biological samples that pertain to two or more different perturbations;
applying a contrastive learning framework on the one or more tuples, to calculate a contrastive loss function that is adapted to (i) minimize variation between perturbation embeddings pertaining to similar perturbations, and (ii) accentuate variation between perturbation embeddings pertaining to different perturbations; and
updating parameters of the ViT model by backpropagating gradients computed from the contrastive loss function.

10. The method of claim 9, wherein each tuple comprises: (i) an anchor image depicting an organelle of a specific type pertaining to a first perturbation; (ii) a positive image depicting another organelle of the same specific type, pertaining to the first perturbation; and (iii) a negative image depicting another organelle of the same specific type pertaining to a second, different perturbation.

11. The method of claim 1, wherein the at least one analysis model comprises a Machine-Learning (ML) based classification model, trained to:
receive one or more perturbation embeddings, originating from said target microscopy images as embedding vectors; and
predict the condition of the biological sample based on said embedding vectors.

12. The method of claim 6, wherein the at least one analysis model is configured to:
combine perturbation embeddings that pertain to different organelle types, but represent a common perturbation, to generate a superposition vector; and
reduce a dimension of the superposition vector, to generate a compact superposition vector, representing an integrated organellome view of a specific biological sample perturbation.

13. The method of claim 12, wherein the at least one analysis model is further configured to:
obtain a plurality of compact superposition vectors, pertaining to a respective plurality of biological samples; and
group the plurality of compact superposition vectors into clusters of a clustering model, wherein each cluster describes a perturbation of respective biological samples.

14. The method of claim 12 further comprising:
obtaining a target compact superposition vector, originating from the one or more target microscopy images of the target biological sample;
associating the target compact superposition vector with a specific cluster of the clustering model, based on a predetermined distance metric; and
determining the condition of the biological sample based on said association.

15. The method of claim 14, further comprising:
identifying a treatment-related perturbation that reverses a disease-related perturbation of a specific disease in a vector space of the clustering model, wherein the treatment-related perturbation is associated with administration of a substance to the biological sample; and
designating the substance for drug development, for treating said specific disease.

16. The method of claim 6, wherein the at least one analysis model comprises an organelle ranking module, configured to:
based on the perturbation embeddings, quantify changes in states of organelles of different types, in response to respective biological sample perturbations; and
rank the organelle types according to discriminatory power between said perturbations, based on said quantification.

17. The method of claim 16, further comprising:
identifying a subset of organelle types with rankings above a predetermined threshold;
analyzing the subset of organelle types to determine their relevance to a specific disease or condition; and
designating at least one organelle type from the analyzed subset as a drug development target based on its relevance to the specific disease or condition and its discriminatory power as indicated by the ranking.

18. A system for determining a condition of a biological sample, the system comprising: a non-transitory memory device, wherein modules of instruction code are stored, and at least one processor associated with the memory device, and configured to execute the modules of instruction code, whereupon execution of said modules of instruction code, the at least one processor is configured to:
receive one or more target microscopy images associated with the biological sample, wherein each microscopy image depicts cellular organelles pertaining to specific organelle types;
infer a Vision Transformer (ViT) model on the one or more target microscopy images, to generate, for each organelle type, a corresponding perturbation embedding, wherein the ViT model is trained using contrastive learning to distinguish between different perturbations of the organelles;
apply at least one analysis model on said perturbation embeddings, to predict the condition of the biological sample.

19. The system of claim 18, wherein applying the at least one analysis model comprises:
combining perturbation embeddings that pertain to different organelle types, but represent a common perturbation, to generate a superposition vector;
reducing a dimension of the superposition vector, to generate a target compact superposition vector, representing an integrated organellome view of the target biological sample perturbation;
obtaining a clustering model comprising a plurality of clusters of compact superposition vectors, originating from a cohort of biological samples, wherein each cluster pertains to a specific perturbation;
associating the target compact superposition vector with a specific cluster of the clustering model, based on a predetermined distance metric; and
determining the condition of the biological sample based on said association.

20. A method of identifying a drug target for treating a disease by at least one processor, the method comprising:
receiving a plurality of microscopy images associated with a cohort of biological samples, wherein each microscopy image depicts cellular organelles pertaining to specific organelle types, and wherein the cohort includes biological samples associated with a healthy state and biological samples associated with a diseased state;
applying a Vision Transformer (ViT) model on the plurality of microscopy images, to generate, for each organelle type, corresponding perturbation embeddings, wherein the ViT model is trained using contrastive learning to distinguish between different perturbations of the organelles;
combining perturbation embeddings that pertain to different organelle types but represent a common perturbation, to generate superposition vectors;
reducing a dimension of the superposition vectors, to generate compact superposition vectors representing integrated organellome views of respective biological sample perturbations;
grouping the compact superposition vectors into clusters of a clustering model, wherein each cluster pertains to a specific perturbation;
calculating a disease-related perturbation vector in a vector space of the clustering model, originating from a cluster associated with the healthy state and terminating at a cluster associated with the diseased state;
identifying a treatment-related perturbation whose superposition vector substantially matches an amplitude of the disease-related perturbation vector, but having an opposite direction, wherein the treatment-related perturbation is associated with administration of a substance to a biological sample; and
designating the substance as a drug target for treating the disease.
